(19) United States
(12) Patent Application Publication
(10) Pub. No.: US 2023/0119521 A1
(43) Pub. Date: Apr. 20, 2023

(54) SURGICAL HANDPIECE WITH ROTATABLE CANNULA HEAD

(71) Applicant: Nova Eye, Inc., Fremont, CA (US)

(72) Inventors: Mark Arnoldussen, Fremont, CA (US); Steve Burnett, Fremont, CA (US); Jordan Green, Kent Town (AU); Victor Previn, Kent Town (AU)

(21) Appl. No.: 17/506,579

(22) Filed: Oct. 20, 2021

(51) Int. Cl. A61F 9/007

(52) U.S. Cl. CPC A61F 9/00781

(57) ABSTRACT

A microsurgical instrument includes a cannula head rotationally coupled to a handpiece. A cannula head connector at a distal end of the handpiece rotationally couples to a handpiece connector at a proximal end of the cannula head. The cannula head connector and handpiece connector rotate coaxially about a longitudinal center axis passing through a cannula extending from the cannula head, providing an adjustable cannula angle between the cannula head and handpiece. The cannula head is securely retained to the handpiece without slippage changing the cannula angle, and the cannula angle is easily changed to a new value by deliberate rotation of the cannula head.

### FIELD OF THE INVENTION

Embodiments are related to surgical instruments having a cannula attached to a handpiece with the cannula configured for insertion into an eye.

### BACKGROUND

A cannula may be inserted into Schlemm's canal or other parts of an eye to deliver microsurgical instruments, viscoelastic fluid, microcannula, stents, or other payloads for reducing intraocular pressure or for other medical purposes. The cannula may be attached to a handpiece configured to advance the cannula and/or payload into a chosen location in an eye. The handpiece may be shaped for a comfortable and secure grip by a surgeon's hand and may include a thumbwheel, finger pad, or other control for positioning the cannula and/or payload passing through the cannula.

A cannula may be formed a straight hollow segment and an arcuate segment near the distal end to facilitate cannula entry into the trabecular meshwork, Schlemm's canal, or other parts of the eye's draining system. The straight segment and arcuate segment together define a plane of curvature for the cannula. For some procedures, canaloplasty of Schlemm's canal being an example, it may be preferable to insert the cannula into eye tissue with the plane of curvature for the cannula parallel to the plane of the approximately circular Schlemm's canal. However, the plane of curvature for the cannula may be held at a fixed angle relative to finger grips and other parts of the handpiece. Adjusting the position of the cannula to place the plane of curvature for the cannula in a preferred orientation relative to an eye therefore requires rotation of the entire handpiece, possibly placing finger grips and payload controls at an inconvenient or fatiguing angle for a surgeon using the handpiece.

### SUMMARY

An example apparatus embodiment includes a handpiece and a cannula head rotatably coupled to the handpiece. The handpiece includes a connector flange extending longitudinally away from a distal end of the handpiece; and an inner flange contact face formed on a side of the connector flange facing toward a longitudinal rotation axis of the handpiece. The cannula head includes a circumferential outer flange extending radially outward from an outer surface of the cannula head; an arcuate inner flange extending longitudinally away from the outer flange; a connector flange contact face formed on a side of the inner flange facing toward the outer flange; and a circumferential channel formed between the outer flange and the inner flange, with the connector flange rotatably engaged in the circumferential channel and the inner flange contact face in sliding contact with the connector flange contact face.

The example apparatus embodiment optionally includes the cannula head retained on the handpiece with a cannula angle in a range from zero degrees (0°) to three hundred sixty degrees (360°).

The example apparatus embodiment optionally includes a value of torque for rotating the cannula head relative to the handpiece in a range from 0.01 Newton-meter to 0.03 Newton-meter.

The example apparatus embodiment optionally further includes the connector flange having an outer projection extending distally away from an exterior distal surface of the handpiece. The outer projection optionally contacts the outer flange when the connector flange is rotatably engaged in the circumferential channel.

The example apparatus embodiment optionally further includes the connector flange having an inner projection extending proximally into a void space formed in said handpiece. The inner flange contact face optionally extends from a distal end of the outer projection to a proximal end of the inner projection.

The inner flange contact face is optionally formed as a cylindrical surface or may alternatively be formed as a frustrum of a cone.

The inner flange optionally includes an inner flange ridge extending radially outward from the connector flange contact face.

The connector flange further optionally includes an inner projection extending proximally into a void space formed in the handpiece. The inner flange ridge contacts the inner projection when the connector flange is engaged in the circumferential channel.

The inner flange further optionally includes an inner flange ridge extending radially away from the connector flange contact face. The inner flange ridge is optionally positioned to press the connector flange against the outer flange.

The example apparatus embodiment optionally includes a cannula extending longitudinally away from a distal end of the cannula head. A longitudinal rotation axis of the handpiece is preferably coaxial with a longitudinal centerline of the cannula. A longitudinal rotation axis of the cannula head is preferably coaxial with a longitudinal centerline of the cannula.

### BRIEF DESCRIPTION OF THE DRAWINGS

FIG. 1 is a view toward the left side of an example microsurgical instrument including a handpiece and a rotatable cannula head.

FIG. 2 is a view toward the top side of the example microsurgical instrument of FIG. 1.

FIG. 3 is a view toward the distal end the example microsurgical instrument of FIG. 1.

FIG. 4 is another view toward the distal end of the example microsurgical instrument of FIG. 1, showing the cannula head removed, a cannula head connector at the distal end of the outer housing of the handpiece, and examples of some mechanical features inside the cuter housing.

FIG. 5 is a partial cross-sectional view A-A of the cannula, cannula head, and a distal portion of the handpiece, showing an example of the handpiece connector on the cannula head engaged with the cannula head connector on the handpiece. A location and viewing direction for the cross-sectional view is indicated with section arrows A in FIG. 2.

FIG. 6 is a partial enlargement of FIG. 5, showing the example cannula head and cannula without the handpiece.

FIG. 7 is another partial enlargement of FIG. 5, showing the example handpiece without the cannula head and cannula.

FIG. 8 is a view toward the distal end of the example microsurgical instrument of FIG. 1, showing an example of an initial position of the cannula plane of curvature.

FIG. 9 continues the example of FIG. 8, showing the cannula head rotated from its initial rotational position in FIG. 8 to a new rotational position.

### DESCRIPTION

Example apparatus embodiments of a microsurgical instrument include a cannula head rotatably retained on a handpiece. A cannula angle for a cannula attached to the cannula head is adjustable with light finger pressure by rotating the cannula head relative to an outer housing of the handpiece. The selected cannula angle remains stable and the cannula head remains firmly in place without slipping relative to the handpiece after the cannula angle is set. The cannula angle may be set, for example, to place a plane of curvature for the cannula in a preferred angular relationship with structures in an eye, or to deliver a payload having a preferred angular orientation into an eye. The handpiece can be held at any rotation angle according to a surgeon's preference, with the cannula angle set according to the requirements of the procedure being performed.

Although the apparatus examples included herein reference a cannula formed from a hollow tubular member having a straight segment and an arcuate segment, apparatus embodiments 100 are equally effective for use with cannulas having an entirely straight hollow tubular member, that is, there is no arcuate segment.

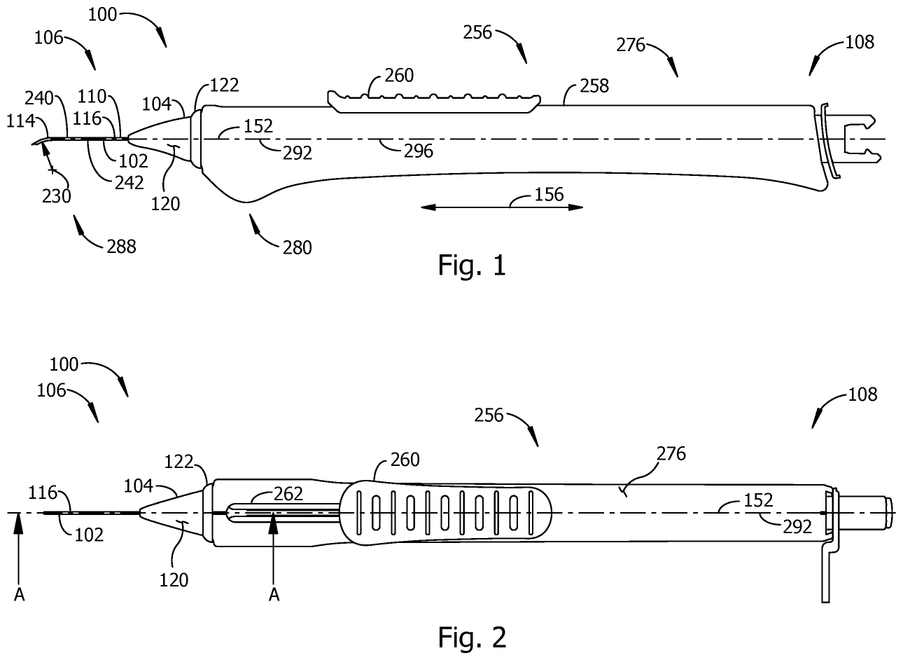

FIGS. 1 and 2 show side on top views of an example apparatus embodiment of the microsurgical instrument 100. The apparatus 100 includes a handpiece 256 having a finger pad 260 slidably engaged on the top side 276 of an outer housing 258. The example finger pad 260 slides in a longitudinal direction 156 in a slot 262. The finger pad 260 can be moved toward the distal end 106 of the handpiece 256 and toward the proximal end of the handpiece to force a payload out of or into a cannula 102 extending from the distal end of the cannula head 104. A finger rest 280 projecting downward from the outer housing 258 on a side opposite the finger pad 260 provides for stable and accurate control of the position of the cannula 102 and a payload extended from the cannula.

The cannula 102 is formed from a hollow tubular member 110 having a straight segment 116 at the proximal end of the cannula and an optional arcuate segment 114 near the distal end of the cannula. A center of curvature 230 for the bend radius of the arcuate segment and the longitudinal centerline 152 of the cannula 102 define a cannula plane of curvature 288. Apparatus embodiments 100 are effective for setting a cannula angle between the cannula plane of curvature and visual and tactile references on the handpiece 256, as will be explained in more detail for later figures. A circumferential outer flange 122 extending radially outward from an outer surface 120 of the cannula head 104 contributes to the cannula head 104 being retained securely to the handpiece while allowing convenient selection of a cannula angle.

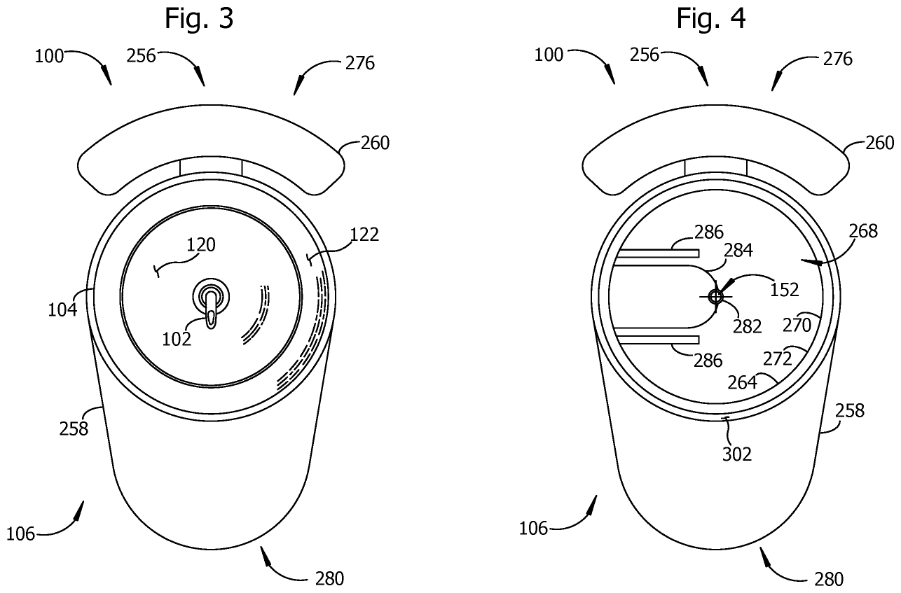

FIG. 3 shows a view toward the distal end 106 of the cannula 102, with the outer flange 122 extending radially outward from the outer surface 120 of the cannula head 104. FIG. 4 shows examples of some internal features of the handpiece located in a void space 268 inside the outer housing 258, including guide rails 236 configured for slidable engagement with parts (not illustrated) of a mechanism coupled to the finger pad 260 for controlling payload movement through the cannula. Other internal features include a hollow payload sleeve 282 attached to a fixed payload sleeve support 284 attached to an interior surface of the outer housing 258. The finger pad 260 may be configured to move part of the payload sleeve to deliver a payload through the cannula. Details of the optional mechanical coupling between the finger pad 260 and the payload sleeve 282 have been omitted.

FIG. 4 further illustrates an example of a cannula head connector 264 configured for rotatable attachment of the cannula head 104. The cannula head connector 264 includes a connector flange 270 having an outer projection 272 extending distally from an exterior distal surface 302 of the outer housing 258. When the handpiece is coupled to the cannula head, the longitudinal rotation axis 292 for the handpiece is collinear and coaxial with the longitudinal centerline 152 of the cannula. The longitudinal centerline 152 of the cannula is also collinear and coaxial with the longitudinal rotational axis of the handpiece connector 235 on the cannula head 104 and the cannula head connector 264 on the handpiece.

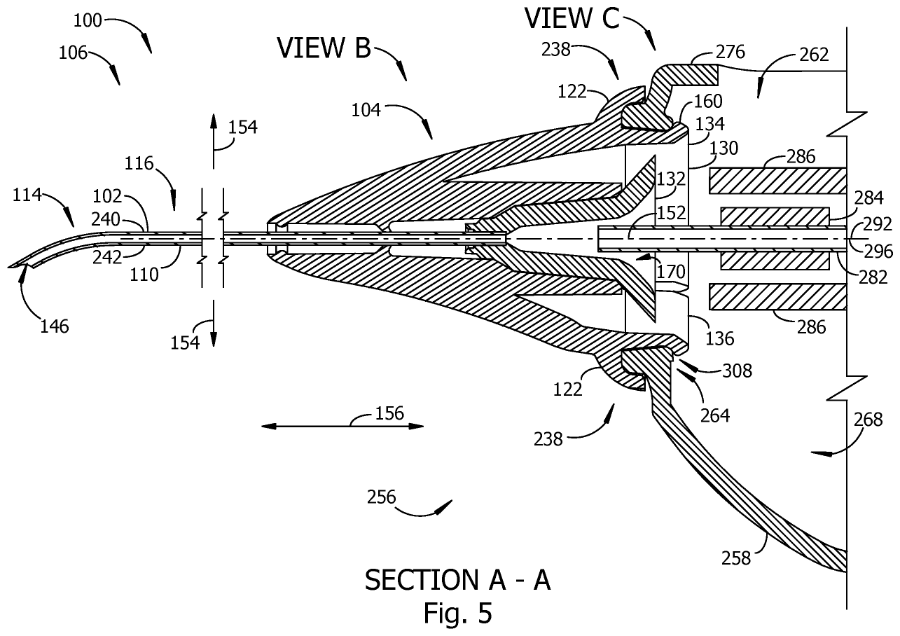

Cross-sectional view A-A in FIG. 5 Shows an example of the engagement of the handpiece connector 238 on the proximal end of the cannula head 104 with the cannula head connector 264 on the distal end of the handpiece outer housing 258. Engagement between the handpiece connector 238 and cannula head connector 264 provides for rotational movement of the cannula head relative to the handpiece while maintaining collinearity of the longitudinal rotation axis 292 of the cannula head connector and the handpiece, the longitudinal rotation axis 296 of the handpiece connector 238 and the cannula head, and the longitudinal centerline 152 of the cannula 102. The longitudinal rotational axis 296 of the cannula head connector corresponds to the longitudinal rotation axis of the cannula head. The collinear centerlines and axes of rotation (152, 292, 296) provide a reference for radial directions 154 used in the figures and establish a common axis for coaxial rotation of the cannula head and handpiece.

The handpiece connector 238 on the cannula head 104 will preferably not slip in rotation relative to the cannula head connector 264 on the handpiece 256 while the cannula is penetrating eye tissue, during delivery of a payload through the cannula, and while the cannula is being removed from an eye, even after exposure of an embodiment 100 to viscoelastic fluid, saline solution, and other fluids that may be present during a medical procedure. However, the cannula head 104 will preferably rotate in response to deliberately applied finger pressure on the handpiece and cannula head, without the use of a wrench or similar tool. Preferred values of rotational torque for rotating the cannula head relative to the handpiece have been measured to fail in a range from 0.01 Newton-meter to 0.03 Newton-meter. Values below 0.01 Newton-meter may allow unintended rotation of the cannula head. Values above 0.03 Newton-meter may increase the difficulty of precisely adjusting the rotational position of the cannula head and/or the cannula angle. For some example embodiments 100 a value of rotational torque for rotating the cannula head relative to the handpiece is 0.015 Newton-meter.

As suggested in FIG. 5, the distal end of the payload sleeve 282 optionally extends into a void space 170 formed by the tapered walls of a payload guide 132 attached to the proximal end of the cannula 102. The payload guide 132 is preferably positioned such that its lumen is in fluid communication with the lumen 146 of the cannula 102 for all cannula angles.

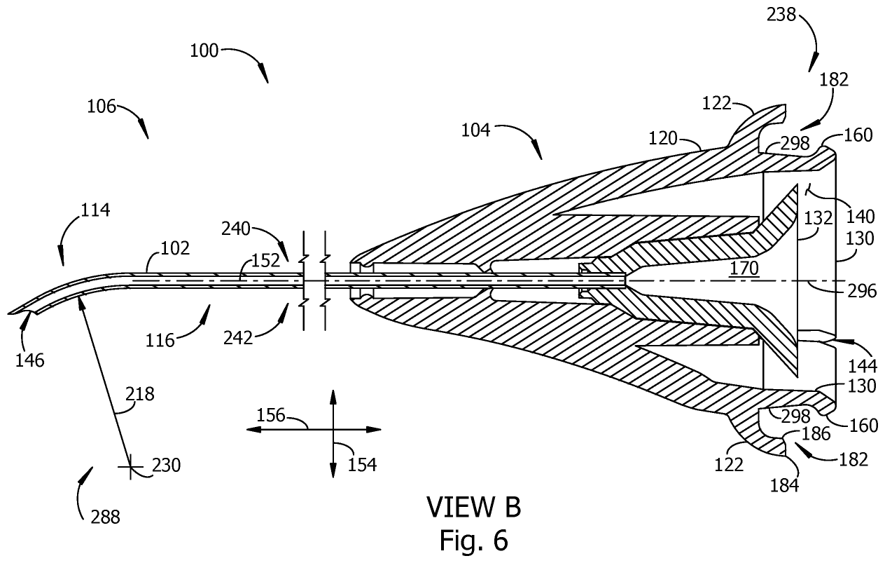

View B in FIG. 6 illustrates examples of some additional features of the handpiece connector 238, The outer flange 122 extends in a radial direction 154 away from the outer surface 120 of the cannula head 104. The outer flange 122 is optionally formed with a circular outer edge 184 and a circular inner edge 186 form the internal and external circular edges of the outer flange 123. The outer flange 122 or other parts of the cannula head 104 may optionally be formed with facets, inset faces, or ridges to enhance tactile feedback during rotation of the cannula head and/or to provide visual indication of the cannula angle.

The inner flange 130 extends longitudinally in a proximal direction 108 from the outer flange 122. The inner flange 130 is formed with an inner surface 140 and a connector flange contact face 298 on a side opposite the inner surface 140. The inner flange 130 may optionally be formed in at least two segments, for example a first inner flange segment 134 and a second inner flange segment 136, with the flange segments separated from one another by intervening inner flange slots 144. Separating the inner flange into segments may enable greater radial flexure of the inner flange, compared to an unsegmented inner flange. Radial flexure of the inner flange may be selected to establish a preferred torque threshold Which must be overcome to initiate rotation of the cannula head relative to the handpiece and a maximum torque required to initiate rotation.

A circumferential inner flange ridge 160 extends in a radial direction 154 outward from the connector flange contact face 298. A circumferential channel 182 is formed between the inner flange ridge 160, connector flange contact face 293, and the inner edge 186 of the outer flange 122. A press fit of the connector flange 270 into the circumferential channel 182 securely retains the cannula head to the handpiece and provides for a value of rotational torque in the preferred range to prevent unintended rotation of the cannula head relative to the handpiece and enable easy rotational adjustment of the cannula angle.

FIG. 6 further illustrates an example of the cannula plane of curvature 288. The optional arcuate segment 114 of the cannula 102 is formed with a bend radius 218 passing through a point marking the miter of curvature 230. The center of curvature 230 and the longitudinal centerline 152 of the straight segment 116 of the cannula 102 are coplanar with one another and establish a plane of curvature 288 for the cannula 102. In the example of View B, the plane of curvature 288 is the plane on which the drawing view is presented. The bottom side 242 of the cannula 102 is the side of the arcuate segment 114 facing the center of curvature 230. The top side 240 of the cannula is the side opposite the bottom side 242.

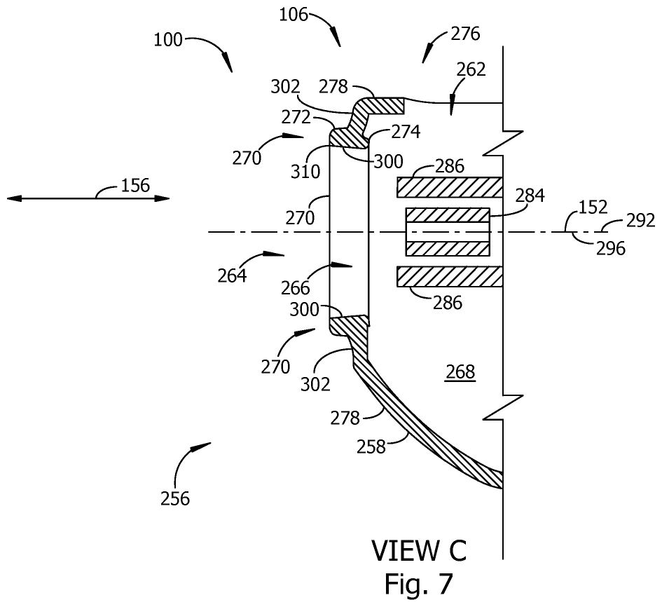

View C in FIG. 7 shows the distal end of the example handpiece 256 from FIG. 5. The example cannula head connector 264 extends in a longitudinal direction 156 from the exterior distal surface 302 of the exterior wall 278 of the handpiece outer enclosure 258. A connector aperture 266 surrounded by the connector flange 270 is in fluid communication with the void space 268 inside the outer enclosure 258. An inner flange contact face 300 on a side of the connector flange 270 facing toward the longitudinal rotation axis 292 is positioned for sliding contact with the connector flange contact face 298 on the inner flange 130 during rotation of the cannula head relative to the handpiece. A circular edge 310 of the inner flange contact face 300 is centered on the longitudinal rotation axis 292 of the handpiece 256.

The connector flange contact face 298 and the inner flange contact face 300 together form a rotational bearing 308 having a rotational axis collinear with the longitudinal centerline 152 of the cannula 102. The rotational bearing 308 establishes coaxial rotation of the cannula 102 and the handpiece connector 238 relative to the cannula head connector 264. In the examples of FIGS. 5-7, the rotational bearing includes opposing faces (298, 300) which are approximately flat. The opposing faces (298, 300) may alternatively be formed with complementary arcuate profiles, for example, one face convex and the other concave.

The connector flange 270 includes an outer projection 272 extending in a longitudinal direction 156 away from the exterior distal surface 302 of the handpiece and an inner projection 274 extending in an opposite longitudinal direction into the void space 268 inside the outer housing 258. The inner flange contact face 300 extends from a distal end of the outer projection 272 to a proximal end of the inner projection 274. In some embodiments, the inner flange contact face 300 and the connector flange contact face 298 on the cannula head are formed as cylindrical surfaces. In other embodiments, the inner flange contact face 300 and the connector flange contact face 298 are formed as the surface of a frustum of a cone. During engagement of the cannula head connector 264 and the handpiece connector 238, the inner flange 130 flexes radially inward toward the cannula longitudinal centerline 152 until the inner flange ridge 160 snaps around the inner projection 274 of the connector flange 270, urging the outer projection 272 of the connector flange into contact with the outer flange 122 of the cannula head 104.

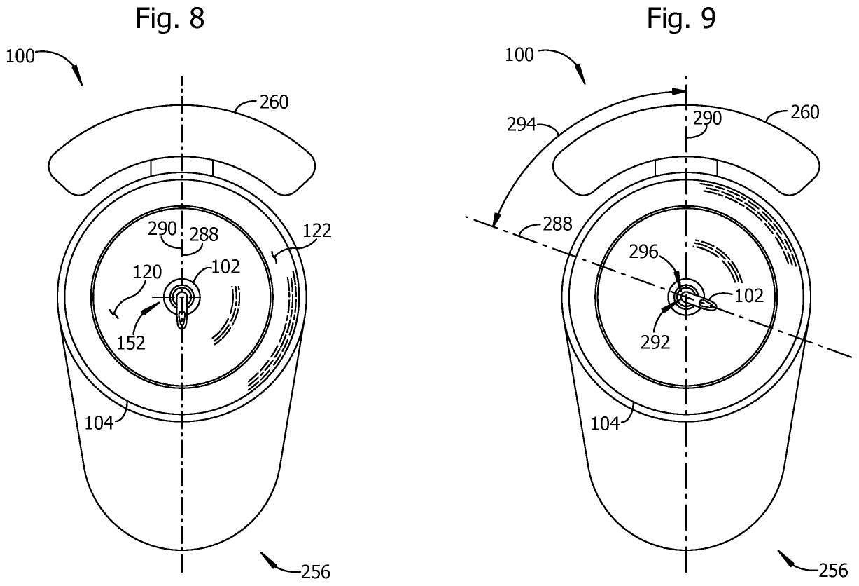

FIG. 8 and FIG. 9 are views of the embodiment 100 toward the outer surface 120 of the cannula head 104. FIG. 8 and FIG. 9 together demonstrate an example of an adjustable cannula angle 294. In the example of FIG. 8, the cannula plane of curvature 288 is shown in an initial position 290. In the example of FIG. 9, the cannula head 104 has been rotated through a cannula angle 294 relative to the initial position 290. The cannula angle 294 may be set to any angle value greater than or equal to zero degrees (i.e., no rotation), and less than or equal to 360 degrees, (i.e., a complete rotation). The cannula angle 294 may optionally be set to values greater than 360 degrees, i.e., more than one full rotation, should a need arise. Rotations may be performed in clockwise and counterclockwise directions at the discretion of the surgeon.

Unless expressly stated otherwise herein, ordinary terms have their corresponding ordinary meanings within the respective contexts of their presentations, and ordinary terms of art have their corresponding regular meanings.

What is claimed is:

1. An apparatus, comprising:
a handpiece comprising:
a connector flange extending longitudinally away from a distal end of said handpiece; and
an inner flange contact face formed on a side of said connector flange facing toward a longitudinal rotation axis of said handpiece; and
a cannula head rotatably attached to said handpiece, comprising:
a circumferential outer flange extending radially outward from an outer surface of said cannula head;
an arcuate inner flange extending longitudinally away from said outer flange;
a connector flange contact face formed on a side of said inner flange facing toward said outer flange; and
a circumferential channel formed between said outer flange and said inner flange, said connector flange rotatably engaged in said circumferential channel with said inner flange contact face in sliding contact with said connector flange contact face.

2. The apparatus of claim 1, wherein said cannula head is retained on said handpiece with a cannula angle in a range from zero degrees (0°) to three hundred sixty degrees (360°).

3. The apparatus of claim 1, wherein a value of torque for rotating said cannula head relative to said handpiece is in a range from 0.01 Newton-meter to 0.03 Newton meter.

4. The apparatus of claim 1, said connector flange further comprising an outer projection extending distally away from an exterior distal surface of said handpiece.

5. The apparatus of claim 4, wherein said outer projection contacts said outer flange when said connector flange is rotatably engaged in said circumferential channel.

6. The apparatus of claim 4, said connector flange further comprising an inner projection extending proximally into a void space formed in said handpiece.

7. The apparatus of claim 6, said inner flange contact face extending from a distal end of said outer projection to a proximal end of said inner projection.

8. The apparatus of claim 7, wherein said inner flange contact face is formed as a cylindrical surface.

9. The apparatus of claim 7, wherein said inner flange contact face is formed as a surface of a frustum of a cone.

10. The apparatus of claim 1, said inner flange further comprising an inner flange ridge extending radially outward from said connector flange contact face.

11. The apparatus of claim 10, said connector flange further comprising an inner projection extending proximally into a void space formed in said handpiece, wherein said inner flange ridge contacts said inner projection when said connector flange is engaged in said circumferential channel.

12. The apparatus of claim 1, said inner flange further comprising an inner flange ridge extending radially away from said connector flange contact face, said inner flange ridge positioned to press said connector flange against said outer flange.

13. The apparatus of claim 1, further comprising a cannula extending longitudinally away from a distal end of said cannula head.

14. The apparatus of claim 13, wherein a longitudinal rotation axis of said handpiece is coaxial with a longitudinal centerline of said cannula.

15. The apparatus of claim 13, wherein a longitudinal rotation axis of id cannula head is coaxial with a longitudinal centerline of said cannula.
